(19) United States
(12) Patent Application Publication
(10) Pub. No.: US 2020/0001567 A1
(43) Pub. Date: Jan. 2, 2020

(54) METHOD FOR MANUFACTURING PLEATED STRETCHABLE COMPOSITE MEMBER, AND PLEATED STRETCHABLE COMPOSITE MEMBER

(71) Applicant: Sun Tool Corporation, 34-15, Dainichi Higashi cho (JP)

(72) Inventor: Shoji HIDAKA, Moriguchi-shi (JP)

(73) Assignee: Sun Tool Corporation, Moriguchi shi, Osaka (JP)

(21) Appl. No.: 16/482,631

(22) PCT Filed: Jan. 29, 2018
(86) PCT No.: PCT/JP2018/003711

§ 371 Date: Jul. 31, 2019

(30) Foreign Application Priority Data
Jan. 31, 2017 (JP) 2017-028459

(51) Int. Cl. B32B 3/28; B32B 5/02; B32B 7/12; B32B 7/022; B32B 37/12; B32B 38/00; A41H 43/02; A61F 13/15; A61F 13/00

(52) U.S. Cl. CPC B32B 3/28; A61F 2013/1591; B32B 7/12; B32B 7/022; B32B 37/1207; B32B 37/1292; B32B 38/0012; A41H 43/0242; A61F 13/15731; A61F 13/15764; A61F 13/00995; B32B 2037/1215; B32B 2038/0028; B32B 2307/726; B32B 2555/02; B32B 5/022

(57) ABSTRACT

In manufacturing a pleated stretchable composite member, a first sheet-like nonwoven fabric and a stretched cord-like elastic member (rubber thread, etc.) are positioned on the protruding surface side of a pattern roll, and a hot melt adhesive is intermittently applied to the stretched cord-like elastic member and the first sheet-like nonwoven fabric at locations where the protruding surface of the pattern roll is present, and then the stretched cord-like elastic member and the first sheet-like nonwoven fabric intermittently coated with the adhesive and a second sheet-like nonwoven fabric are integrally adhesively fixed at positions corresponding to locations where the protruding surface of the pattern roll is present. In the stretchable composite member, the valleys of the upward-facing breathers of the top-layer nonwoven sheet and the valleys of the downward-facing breathers of the bottom-layer nonwoven sheet are adhesively fixed via a stretchable elastic members (rubber threads) between the two nonwoven fabric sheets.

### TECHNICAL FIELD

The present invention relates to a stretchable composite member which can form pleats (gathers) that are pliant, soft and feel good to the touch and to a method for manufacturing the same.

### BACKGROUND ART

In absorbent products such as disposable diapers and sanitary napkins, it is common practice to join an elastic member in a stretched state to a sheet material and allow the elastic member to contract, thereby forming pleats (gathers) in the sheet material.

There are disposable diapers that include an elastic stretch portion having a linear joint portion of sheet members extending in a direction perpendicular to the stretching direction of the elastic member, and in which the elastic members are joined to the sheet members at the joint portions. There are also stretchable composite members for filtering use in which a large number of pleats are formed in the lateral direction.

### PATENT LITERATURE

Patent Literature 1: Japanese Utility Model Application Laid-Open (Kokai) No. H4-71922
Patent Literature 2: Japanese Patent No. 5,928,469

### DISCLOSURE OF INVENTION

### Problems to be Solved by the Invention

Patent Literature 1 (title: Gathers for Disposable Product) is a stretchable composite member having two sheet materials and a plurality of elastic members disposed between the two sheet materials, wherein the sheet materials are partially joined together to form a plurality of joint portions, the plurality of joints form a plurality of joint lines extending in a direction intersecting the stretching direction of the elastic members, each of the elastic members R is fixed between the two sheet materials in at least a part of the joint portions HML, and each of the two sheet materials forms pleats (gathers) F between the adjacent joint lines (see FIG. 27).

Also, there is a known stretchable composite member that serves as a filter element, in which pleats (gathers) F and grooves GV are formed by a stretchable composite member having two sheets, namely, a stretchable nonwoven fabric AF and a non-stretchable nonwoven fabric CF, and a plurality of elastic members R disposed between these two sheet materials (see FIG. 28).

Patent Literature 2 (Roll Transfer Application Method and Application Device for Hot Melt Adhesive; hereinafter referred to as a previous invention) discloses a roll transfer application method of a hot melt adhesive, which was invented by the inventor of the invention of the present patent application and patented to the applicant of the present application; and this method includes a step of transferring a hot melt adhesive, which has been applied to the protruding portions of a pattern roll, from the protruding surfaces of the pattern roll to the surface of a coating substrate, wherein

a coater head whose slot groove runs in the axial direction of the pattern roll is provided to face the pattern surfaces of the pattern roll, and the hot melt adhesive supplied from a hot melt adhesive supply device is supplied in a thin film state to the pattern surfaces of the pattern roll, so that a thin film of hot melt adhesive is supplied to only the protruding surfaces of the pattern roll.

The object of the present invention is to apply the above-mentioned previous invention in the manufacture of a pleated stretchable composite member so as to obtain a stretchable composite member that is pliant, soft, and feels good to the touch, and to securely fix a first sheet-like nonwoven fabric, a stretched cord-like elastic member, and a second sheet-like nonwoven fabric.

The first invention of the present application provides a method for manufacturing a stretchable composite member, wherein a coater head whose slot grooves run in the axial direction of a pattern roll is provided to face the pattern surfaces of the pattern roll, and a hot melt adhesive supplied from a hot melt adhesive supply device is supplied in a thin film state to the pattern surfaces of the pattern roll, thereby supplying a thin film of the hot melt adhesive to only the protruding surface of the pattern roll;

in the step of transferring the thin film of hot melt adhesive applied to only the protruding surface of the pattern roll from the protruding surfaces of the pattern roll to the surface of a coating substrate,
a first sheet-like nonwoven fabric and a stretched cord-like elastic member (rubber thread, etc.) are positioned on the protruding surface side of the pattern roll, the hot melt adhesive is intermittently applied in the travel direction of the stretched cord-like elastic member to the stretched cord-like elastic member and the first sheet-like nonwoven fabric at locations where the protruding surfaces of the pattern roll is present; and

in the subsequent nip roll step,
the stretched cord-like elastic member and the first sheet-like nonwoven fabric, on which intermittent coatings are done in the previous step, and a second sheet-like nonwoven fabric are adhesively fixed, and
the first sheet-like nonwoven fabric and the second sheet-like nonwoven fabric are integrally adhesively fixed along with the hot melt adhesive intermittently applied to the stretched cord-like elastic member at positions corresponding to locations where the protruding surfaces of the pattern roll are present;

so that the integrally fixed area of the first sheet-like nonwoven fabric, the stretched cord-like elastic member, and the second sheet-like nonwoven fabric is intermittently distributed so as to correspond to the configuration of the protruding surfaces.

The second invention of the present application provides a method for manufacturing a pleated stretchable composite member, wherein a coater head whose slot groove runs in the axial direction of a pattern roll is provided to face the pattern surface of the pattern roll, and a hot melt adhesive supplied from a hot melt adhesive supply device is supplied in a thin film state to the pattern surfaces of the pattern roll, thereby supplying a thin film of hot melt adhesive to only the protruding surface of the pattern roll;

in the step of transferring the thin film of hot melt adhesive applied to only the protruding surface of the pattern roll from the protruding surface of the pattern roll to the surface of a coating substrate,
the protruding surfaces of the pattern roll are in a linear form in a direction perpendicular to the rotation direction of the pattern roll,
a first sheet-like nonwoven fabric and a stretched cord-like elastic member are supplied to the transfer position of the protruding surfaces of the pattern roll, and
the cord-like elastic member is contact-guided to the lower face of a guide rail provided between the supplied first sheet-like nonwoven fabric and the supplied cord-like elastic member, so that only the cord-like elastic member is brought into contact with the protruding surfaces of the pattern roll in front of the transfer position and so that the thin film hot melt adhesive is supplied and applied to only the stretched cord-like elastic member before the hot melt is supplied and applied to the first sheet-like nonwoven fabric; and

in the subsequent nip roll step, the stretched cord-like elastic member and the first sheet-like nonwoven fabric intermittently applied in the above step and a second sheet-like nonwoven fabric are adhesively fixed,
the first sheet-like nonwoven fabric and the second sheet-like nonwoven fabric are integrally adhesively fixed in a dot pattern along with the hot melt adhesive intermittently applied to the stretched cord-like elastic member at positions corresponding to locations where the protruding surfaces of the pattern roll are present;

so that the integrally fixed area of the first sheet-like nonwoven fabric, the stretched cord-like elastic member, and the second sheet-like nonwoven fabric is intermittently distributed in a linear configuration in a direction perpendicular to the travel direction of the stretched cord-like elastic member.

The third invention of the present application provides a stretchable composite sheet in which a plurality of stretchable elastic members (rubber threads) are adhesively fixed between two nonwoven fabric sheets in which numerous breathers (gathers) are formed in the lateral direction, thereby forming two layers of breathers (gathers), wherein
the two layers of breathers (gathers) are located one above the other, with the valleys of the upward-facing breathers (gathers) of the top-layer nonwoven fabric sheet and the valleys of the downward-facing breathers (gathers) of the bottom-layer nonwoven fabric sheet being adhesively fixed in a two-dimensional layout via the plurality of stretchable elastic members (rubber threads) between the two nonwoven fabric sheets; and

the fixed area produced by the adhesive fixing is a planar configuration of the pattern distribution that corresponds to the distribution of the protruding surfaces of the pattern roll.

The fourth invention of the present application provides a stretchable composite sheet in which a plurality of stretchable elastic members (rubber threads) are adhesively fixed between two nonwoven fabric sheets in which numerous breathers (gathers) are formed in the lateral direction, thereby forming two layers of breathers (gathers), wherein

the two layers of breathers (gathers) are located one above the other, with the valleys of the upward-facing breathers (gathers) of the top-layer nonwoven fabric sheet and the valleys of the downward-facing breathers (gathers) of the bottom-layer nonwoven fabric sheet being adhesively fixed in a two-dimensional layout via the plurality of stretchable elastic members (rubber threads) between the two nonwoven fabric sheets; and

the fixed area produced by the dot adhesive fixing is a planar configuration of the pattern distribution that corresponds to the distribution of the protruding surfaces of the pattern roll and is expanded around the application area corresponding to the protruding surfaces of the pattern roll in top view.

### Effects of the Invention

The present invention provides a stretchable composite member in which the integrally fixed area comprising the first sheet-like nonwoven fabric, the stretched cord-like elastic member, and the second sheet-like nonwoven fabric is intermittently distributed so as to correspond to the configuration of the protruding surfaces of the pattern roll. Accordingly, the stretchable composite member is flexible, and it is pliant, soft, and feels good to the touch. In addition, the first sheet-like nonwoven fabric, the stretched cord-like elastic member, and the second sheet-like nonwoven fabric are securely fixed together, so that the present invention has the effect of enhancing the product value of a stretchable composite member.

### BRIEF DESCRIPTION OF THE DRAWINGS

FIG. 1 A front view showing the main parts of a roll transfer coating device for a hot melt adhesive according to the previous invention.

FIG. 2 A vertical cross-sectional view of a coater head.

FIG. 3 A lateral cross-sectional view of the same.

FIG. 4 A partial top view of the pattern roll surface, showing various embodiments of protruding patterns of the pattern roll.

FIG. 5 Diagrams of the principle of the present invention, illustrating the timing of applying the hot melt adhesive to the pattern roll surface from the coater head, with FIG. a being a simplified cross-sectional view and FIG. b a partial top view.

FIG. 6 Similarly, diagrams of the timing of the completion of coating the protruding portions, with FIG. 6 being a simplified cross-sectional view and FIG. b a partial top view.

FIG. 7 A front view showing the main parts of a roll transfer coating device for a hot melt adhesive carrying out to implement the first invention of the present application.

FIG. 8 A top view of the same.

FIG. 9 A comb-shaped rotary guide, with FIG. a being a front view, FIG. b a vertical cross-sectional view, and FIG. c a side view.

FIG. 10 A comb-shaped stationary guide, with FIG. a being a front view and FIG. b a cross-sectional view.

FIG. 11 A distribution state of the fixed area according to the present invention, with FIG. a being a front view and FIG. b a top view.

FIG. 12 A detail cross-sectional view of the fixed area, with FIG. a showing the transfer position and FIG. b the position of adhesion to the second coating substrate.

FIG. 13 A simplified view of a roll transfer coating device for a hot melt adhesive for carrying out the second invention of the present application.

FIG. 14 A partial top view of the same.

FIG. 15 A comb-shaped rotary guide, with FIG. a being a front view, FIG. b a vertical cross-sectional view, and FIG. c a side view.

FIG. 16 A comb-shaped stationary guide, with FIG. a being a front view and FIG. b a cross-sectional view.

FIG. 17 A distribution state of the fixed area according to the second invention of the present application, with FIG. a being a front view and FIG. b a side view.

FIG. 18 A detailed partial cross-sectional view of the fixed area, with FIG. a showing at a transfer position and FIG. b at a position of adhesion with the second coating substrate.

FIG. 19 A perspective view of a pleated stretchable composite member, showing a first embodiment according to the present invention.

FIG. 20 A pattern distribution corresponding to a first embodiment of the stretchable composite member of the present invention (partial top view of the pattern roll surface), for the pattern distribution of the protruding portions of the pattern roll shown in FIG. 4 (partial top view of the pattern roll surface).

FIG. 21 Similarly, a reference view showing an embodiment of an individual protruding surface.

FIG. 22 A perspective view of a pleated stretchable composite member, showing a second embodiment according to the present invention.

FIG. 23 A pattern distribution corresponding to the second embodiment (a developed partial top view of the pattern roll surface).

FIG. 24 A perspective view of a pleated stretchable composite member, showing a third embodiment according to the present invention.

FIG. 25 A pattern distribution corresponding to the third embodiment (a developed partial top view of the pattern roll surface).

FIG. 26 A pattern distribution in another embodiment (a developed partial top view of the pattern roll surface).

FIG. 27 A perspective view of a pleated stretchable composite member based on Patent Literature 1.

FIG. 28 A perspective view of another known pleated stretchable composite member.

### BEST MODE FOR CARRYING OUT THE INVENTION

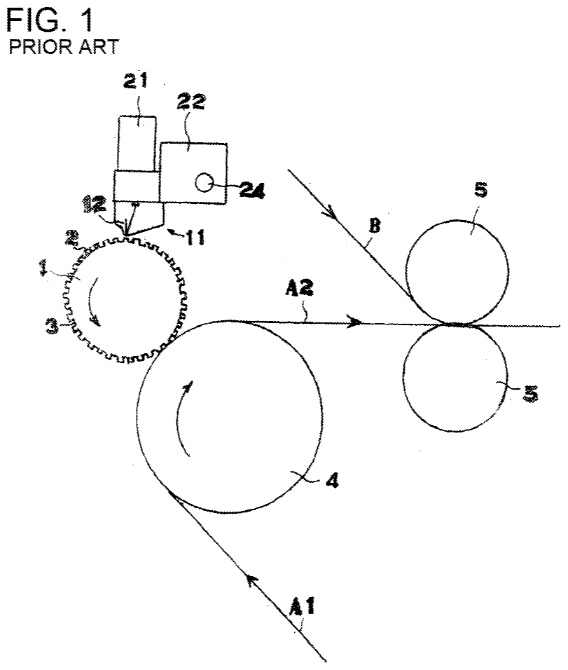

Before describing the present invention, the previous invention (Patent Literature 2), the “Roll Transfer Application Method and Application Device for Hot Melt Adhesive.” The roll transfer coating device for a hot melt adhesive according to the previous invention will be described with reference to FIGS. 1 to 4.

A hot melt adhesive HM supplied from a hot melt adhesive supply device 10 is applied to protruding surfaces 2 of the pattern surface of a pattern roll 1 on which protruding surfaces 2 and recessed surfaces 3 are formed. A coating substrate M1 is clamped between the pattern roll 1, on which the protruding and recessed portions are formed, and an impression cylinder roll 4, and the coating substrate is moved while pressure contact is maintained. The hot melt adhesive HM applied to the protruding surfaces 2 of the pattern roll is transferred from the protruding surfaces 2 onto the surface of the coating substrate A1 with the coating substrate A1 clamped between the pattern roll 1 and the impression cylinder roll 4 and moving while in pressure contact. A coated substrate A2 whose surface has been coated with an adhesive is affixed to a bonding material B by a pair of nip rolls 5.

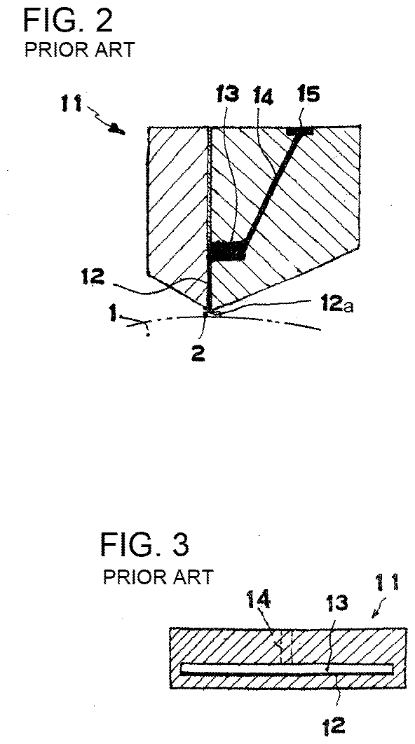

The hot melt adhesive supply device 10 is equipped with a coater head 11 shown in FIGS. 2 and 3. The coater head 11 communicates with an opening 12a at the lower end of a slot groove 12 and an adhesive inlet 15 at the upper end of a supply path 14 via the slot groove (adhesive hole) 12, a supply chamber 13, and the supply path 14. The adhesive inlet 15 communicates with a hot melt adhesive supply body 7 including an adhesive tank 8 and a pressure pump 9 via a valve mechanism 6. The slot groove (adhesive hole) 12 is in the form of a slit with a narrow cross section in the axial direction of the pattern roll. The opening 12a at the lower end of the slot groove 12 faces the pattern surface of the pattern roll 1, with a slight gap being formed between the opening 12a and the protruding surface 2 of the pattern roll 1.

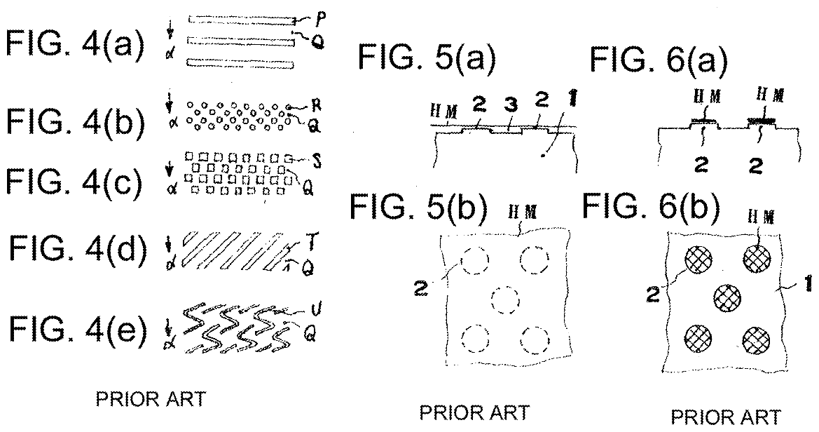

FIG. 4 is a diagram of protruding surface patterns, and shows examples of the distribution of protruding surfaces 2 of the pattern roll 1. In the (a) stage, protruding straight lines P and recessed straight lines (blank lines) Q are provided alternately in the coating travel line direction (α). The same applies to a direction (the lengthwise direction of the slit groove) perpendicular to the coating travel line direction (α). That is, this is a pattern in which the protruding straight lines P are present at set intervals in the coating travel line direction (α). In the (b) stage, protruding circles R are arranged in a zigzag pattern, and in the (c) stage, protruding squares S are arranged in a grid pattern. In both cases, recessed portions (blank portions) Q are present in both the coating travel line direction (α) and the perpendicular direction (the lengthwise direction of the slit groove). In the (d) and (e) stages, continuously repeating patters T and U, in which protruding portions and recessed portions alternate, are formed in the coating travel line direction (α).

Next, the coating action according to the previous invention will be described.

The principle of the previous invention is that the slot groove (adhesive hole) 12 of the coater head is formed as an extremely narrow (being set according to the thickness of a shim) slit that runs perpendicular to the substrate conveyance direction, and the hot melt adhesive supplied from the slot groove forms a coating over the entire surface of the pattern roll in an exceedingly thin thickness (5μ to 30μ) (see FIG. 6).

Next, the hot melt adhesive facing the recessed portions of the pattern roll is absorbed into the hot melt adhesive that faces and adheres to the protruding portions of the pattern roll and integrated therein. As a result, the hot melt adhesive is applied only to the protruding surfaces, and the hot melt adhesive does not adhere to the recessed portions. In other words, the hot melt adhesive is applied only to the surfaces of the protruding portions (see FIG. 6).

FIGS. 5 and 6 illustrate a case in which the entire surface of the pattern roll is coated with a very thin film (5μ to 30μ). When the slot groove (adhesive hole) 12 of the coater head is even narrower (such as when the thickness of a shim inserted between the left and right halves of the coater head to form the slot groove (adhesive hole) 12 of the coater head is 0.2 mm), then if the protruding surfaces are touching the opening of the slot groove (adhesive hole) 12 of the coater head, or if the spacing between them is extremely small, then the amount of adhesive flowing out of the slot groove (adhesive hole) 12 of the coater head will be small, resulting in an oozing state, and out of the surfaces of the pattern roll, the hot melt adhesive will adhere only to the surfaces of the protruding portions and will not adhere to the recessed portions.

The position where the coater head is set is freely selected from among 360 degrees around the outside of the pattern roll, so in this case it is effective to set the opening of the coater head upward.

In working the present invention, if a large quantity of hot melt adhesive is discharged, such as when the total area of the surfaces of the recessed portions 3 of the pattern roll is increased, there is a risk that the hot melt adhesive will adhere to the surfaces of the recessed portions 3.

To deal with this, it is necessary to reduce the discharge amount of the hot melt adhesive.

One way to reduce the discharge amount of the hot melt adhesive HM2 is to increase the rotational speed of the pattern roll to correspond to an increase in the total area of the recessed pattern surface of the pattern roll as compared to the total area of the protruding pattern surface of the pattern roll. As a result, the increase in the recessed pattern surface of the pattern roll is offset by the decrease in the discharge time period of the hot melt adhesive.

Furthermore, the discharge amount of the hot melt adhesive can be varied by changing the conditions below, and the settings can be appropriately selected.
1. Adjustment of the rotational speed of a pump used to supply adhesive in the hot melt adhesive supply device for the adhesive discharge amount of a slot die.
2. Adjustment of the tiny gap between the protruding pattern surface of the pattern roll and the opening of the adhesive hole in the slot die.
3. Adjustment of the temperature of the hot melt supply path in the hot melt adhesive supply device.
4. Adjustment of the cross-sectional width of adhesive in the slot die.

### Effective Example 1

Pattern of protruding and recessed portions: Linear protruding portions arranged at regular intervals in the rotational direction

Full width in the axial direction

Protruding portions and recessed portions alternate in the rotational direction, and the spacing between the protruding portions and recessed portions is 7.2 mm

Width of protruding portions is 1 mm

Height of protruding portions is 1 mm

Coating surface of coating substrate: Coating in a straight line of 1 mm width at intervals of 7.2 mm in the travel direction of the coating lines

Film thickness of coated surface: 5μ to 30μ

Slit (adhesive hole) opening on bottom of slot die: 90 mm×0.2 mm rectangle

Rotational speed of pump used to supply adhesive in hot melt adhesive supply device: 19 rpm

Spacing between protruding pattern surfaces of pattern roll and opening of adhesive hole of slot die: 0.015 to 0.05 mm

Temperature of hot melt supply path in hot melt adhesive supply device: 160° C.

Temperature of pattern roll: 130° C. to 60° C.

### Effective Example 2

Pattern of protruding and recessed portions: Circular protruding portions arranged in a grid pattern

Protruding portions and recessed portions alternate in the axial direction, and the spacing between the protruding portions and recessed portions is 4 mm

Protruding portions and recessed portions alternate in the rotational direction, and the spacing between the protruding portions and recessed portions is 5 mm

Diameter of protruding portions: 2 mm

Height of protruding portions: 1 mm

Surface of coating substrate: Coating in a straight line of 40 mm width at intervals of 5 mm in the travel direction of the coating lines

Film thickness of coated surface: 5μ to 30μ

Slit (adhesive hole) opening on bottom of slot die: 90 mm×0.2 mm rectangle

Rotational speed of pump used to supply adhesive in hot melt adhesive supply device: 10 rpm

Spacing between protruding pattern surfaces of pattern roll and opening of adhesive hole of slot die: 0.015 to 0.05 mm

Temperature of hot melt supply path in hot melt adhesive supply device: 160° C.

Temperature of pattern roll: 130° C. to 65° C.

The effective examples given above are examples of coating conditions, and it should be noted that they do not indicate limiting conditions.

Next, an example of the coating conditions of a coating failure will be given.

### Reference Example 1

In Embodiment 1, at rotational speed of a pattern roll of 38 rpm, part of the hot melt adhesive touched the surfaces of recessed portions, resulting in coating failure.

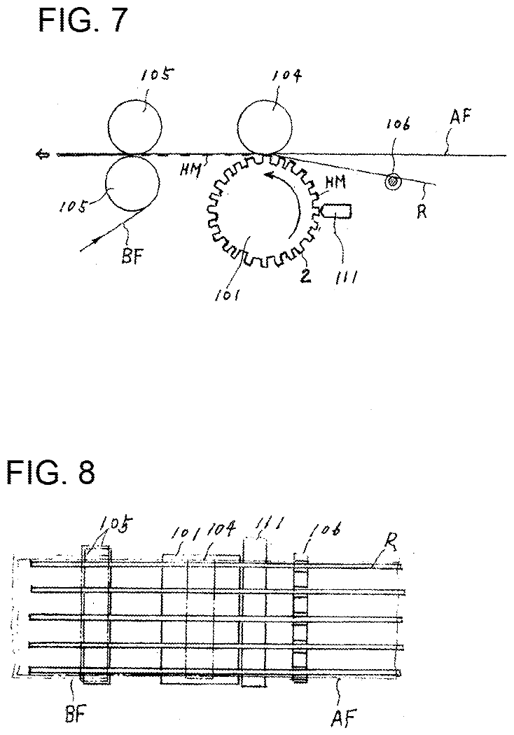

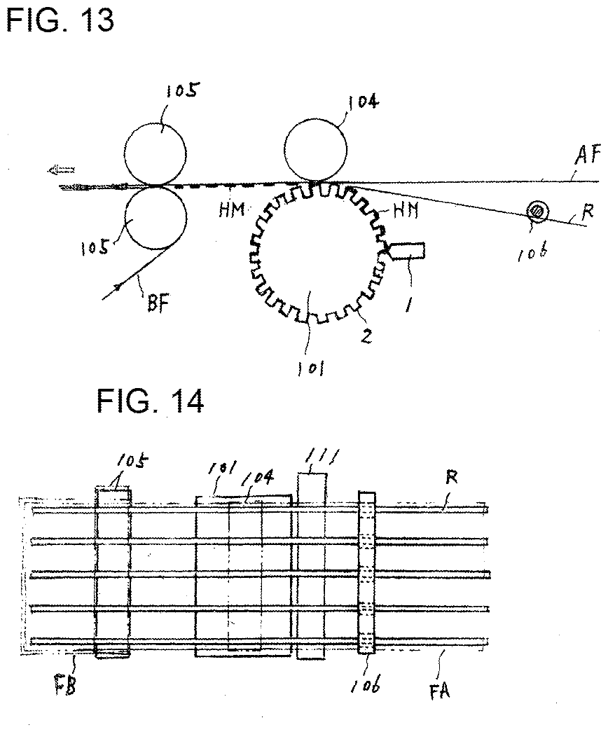

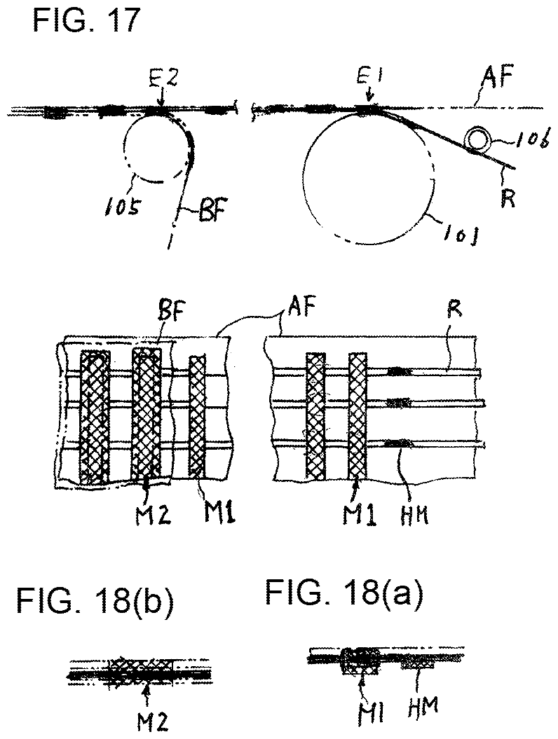

Next, an embodiment of the present invention will be described in detail with reference to FIGS. 7 to 19.

FIG. 7 shows a roll transfer coating device for a hot melt adhesive used in an embodiment of the first invention of the present application, and it is equipped with a pattern roll 101, an impression cylinder roll (rolling roll) 104, nip rolls 105, and a coater head 111, which are the same as the pattern roll 1, impression cylinder roll (rolling roll) 4, nip rolls 5, and coater head 11 in the roll transfer coating device for a hot melt adhesive according to the previous invention shown in FIG. 1.

The coater head 111, whose slot groove runs in the axial direction of the pattern roll 101, is provided to face the pattern surface of the pattern roll 101, and a hot melt adhesive supplied from a hot melt adhesive supply device is supplied in a thin film state to the pattern surfaces of the pattern roll 101.

The thin film of hot melt adhesive HM supplied to the pattern surface of the pattern roll 101 adheres only to the protruding surfaces 2 of the pattern roll 101 and moves to the transfer position.

Here, for the pattern roll 101, and for the protruding surfaces 2, the (a) stage in FIG. 4 is applied, resulting in a linear configuration at a right angle to the rotation direction (a rectangle of narrow width).

For the protruding surfaces 2, the (a) stage in FIG. 4 is applied, so that the distribution state of the hot melt adhesive HM in a thin film state is as shown in a developed view at the (a) stage in FIG. 4. That is, with respect to the coating substrate A, coating is performed intermittently in a linear configuration (a rectangle of narrow width) in a direction perpendicular to the travel direction.

In working the present invention, the following configuration is added to the previous invention described above.

The first sheet-like nonwoven fabric AF and the stretched cord-like elastic member R are supplied as the first coating substrates of the previous invention between the pattern roll 101 and the impression cylinder roll (rolling roll) 104, that is, supplied to the transfer position E1 of the pattern roll 101.

In the subsequent nip roll step, the second sheet-like nonwoven fabric BF is supplied, as the second coating substrate of the previous invention, between the nip roll 105 and the nip roll 105, which is a pressure contact position E2 produced by the two rolls.

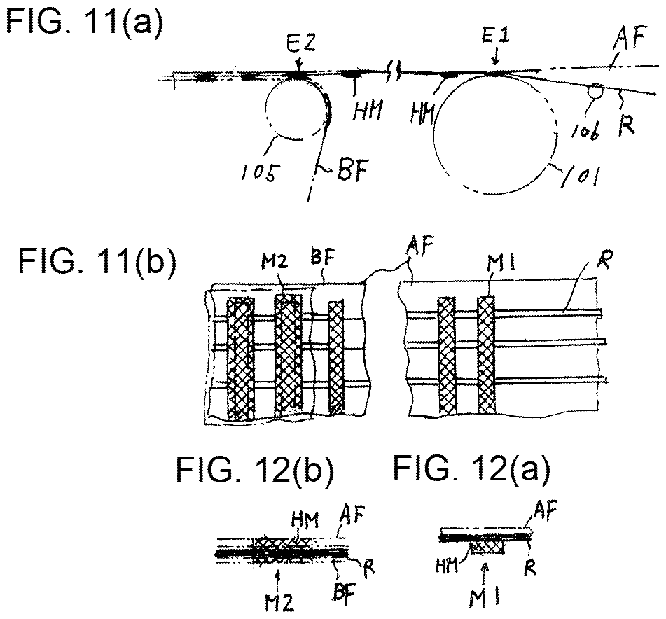

In supplying the first sheet-like nonwoven fabric AF and the stretched cord-like elastic member R, which make the first coating substrate, the stretched cord-like elastic member R is positioned on the lower side of the first sheet-like nonwoven fabric AF (or the protruding surface side of the pattern roll). That is, a thin film of the hot melt adhesive HM is supplied with the stretched cord-like elastic member R being on the lower side, the cord-like elastic member R and the first sheet-like nonwoven fabric AF are bonded by the hot melt adhesive HM (see FIGS. 11 and 12).

In the next nip roll step, the second sheet-like nonwoven fabric BF (the second coating substrate) is adhesively fixed to the stretched cord-like elastic member R and the first sheet-like nonwoven fabric AF serving as the first coating substrate as a result of the above step.

As a result, the first sheet-like nonwoven fabric AF and the second coating substrate BF are integrally adhesively fixed along with the stretched cord-like elastic member R at positions corresponding to locations where the protruding surface of the pattern roll is present, forming the fixed area M2 shown in FIG. 8.

As shown in FIG. 7, the integrally fixed area M2 is intermittently distributed so as to correspond to the configuration of the protruding surfaces, so that this area is intermittently present in the configuration of bars in a direction perpendicular to the cord-like elastic member R.

In the shown embodiment, as shown in FIGS. 7 to 10, a comb-shaped guide 106 is provided in front of the transfer position E1. With its guide grooves, this comb-shaped guide 106 regulates the spacing between the supplied elastic cords (rubber threads) R, with the upper face of the comb-shaped guide 106 used as a guide surface.

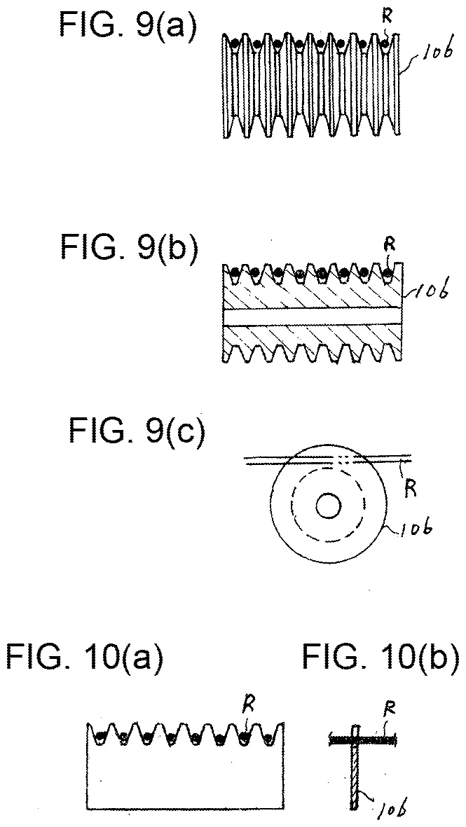

As examples of the comb-shaped guide 106, FIG. 9 shows a comb-shaped rotary guide, and FIG. 10 shows a comb-shaped stationary guide.

In the next nip roll step, the second sheet-like nonwoven fabric BF (the second coating substrate) is adhesively fixed to the stretched cord-like elastic member R and the first sheet-like nonwoven fabric AF serving as the first coating substrate as a result of the above step.

As a result, the first sheet-like nonwoven fabric AF and the second coating substrate BF are integrally adhesively fixed along with the stretched cord-like elastic member R at positions corresponding to locations where the protruding surface of the pattern roll is present, forming the fixed area M2 shown in FIG. 8.

As shown in FIG. 7, the integrally fixed area M2 is intermittently distributed so as to correspond to the configuration of the protruding surfaces, so that this area is intermittently present in the configuration of bars in a direction perpendicular to the cord-like elastic member R.

Next, an embodiment of the second invention will be described with reference to FIGS. 11 to 18.

In this embodiment, referring to FIGS. 14 to 18, in supplying the first sheet-like nonwoven fabric AF and the stretched cord-like elastic member R serving as the first coating substrates, the stretched cord-like elastic member R is positioned on the lower side (on the protruding surface side of the pattern roll). In other words, a thin film of the hot melt adhesive HM is supplied in a state in which the elongated elastic member R is on the lower side, and the hot melt adhesive HM is bonded to the cord-like elastic member R and the first sheet-like nonwoven fabric AF, which is the same as in the embodiment described above (see FIGS. 11 and 12).

In working the second invention, in the embodiment, referring to FIGS. 7 to 10, the guide groove of the comb-shaped guide 106 provided in front of the transfer position E1 uses the lower face of the comb-shaped guide 106 as a guide surface for the supplied cord-like elastic cords (rubber threads) R.

As examples of the comb-shaped guide 106, FIG. 9 shows a comb-shaped rotary guide, and FIG. 10 shows a comb-shaped stationary guide.

The action of the comb-shaped guide 106 causes the elastic cords (rubber threads) R to touch the protruding surfaces 2 of the pattern roll prior to the transfer position E1. As a result, the thin film of hot melt adhesive HM is supplied and applied only to the stretched cord-like elastic member prior to transfer and ahead of the first sheet-like nonwoven fabric.

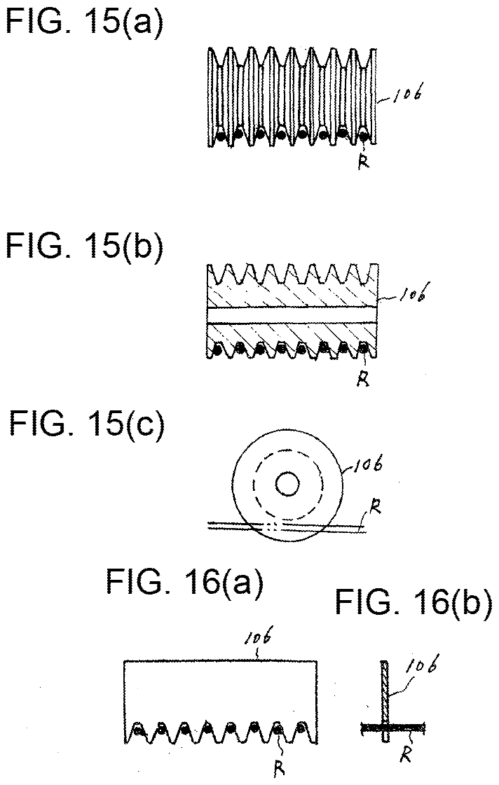

As shown in FIG. 15, after the thin film of hot melt adhesive is intermittently applied to the stretched cord-like elastic member R, the hot melt adhesive is transferred and coated to the first sheet-like nonwoven fabric AF at the transfer position, forming the fixed area M′ shown in FIG. 16.

After this, in the nip roll step, the second sheet-like nonwoven fabric B is pressed against the lower face side of the stretched cord-like elastic member R and the first sheet-like nonwoven fabric A that have been intermittently coated with the thin film of hot melt adhesive HM.

The first sheet-like nonwoven fabric A, the stretched cord-like elastic member R, and the second sheet-like nonwoven fabric B are integrally adhesively fixed in the fixed area M over the range that faces the protruding surfaces 2.

Referring to FIG. 16, in the fixed area F, the first sheet-like nonwoven fabric A and the second sheet-like nonwoven fabric B are in contact with each other at locations where there is no cord-like elastic member R, and the first sheet-like nonwoven fabric A and the second sheet-like nonwoven fabric B are fixed to each other via the hot melt adhesive HM.

FIG. 19 shows the pleated stretchable composite member of the present invention, in which two layers of breathers (gathers) are placed one above the other, and the valleys of the upward-facing breathers (gathers) of the top-layer nonwoven fabric sheet and the valleys of the downward-facing breathers (gathers) of the bottom-layer nonwoven fabric sheet adhesively are fixed in a two-dimensional dot layout with a plurality of stretchable elastic members (rubber threads) in between the two nonwoven fabric sheets.

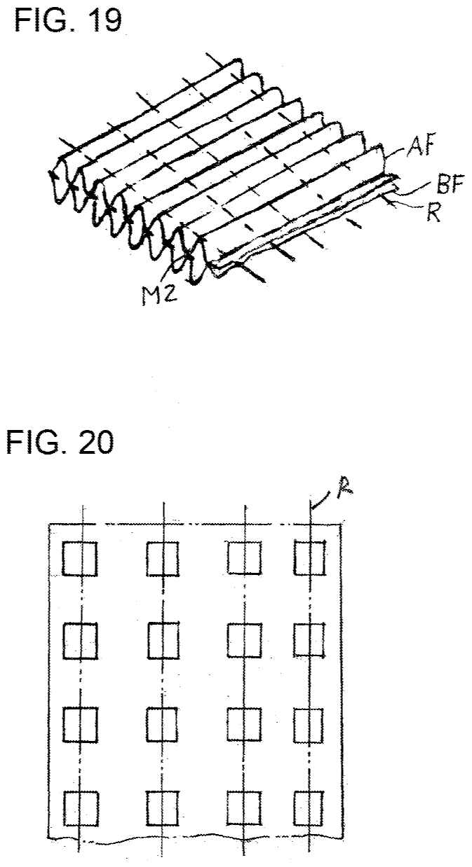

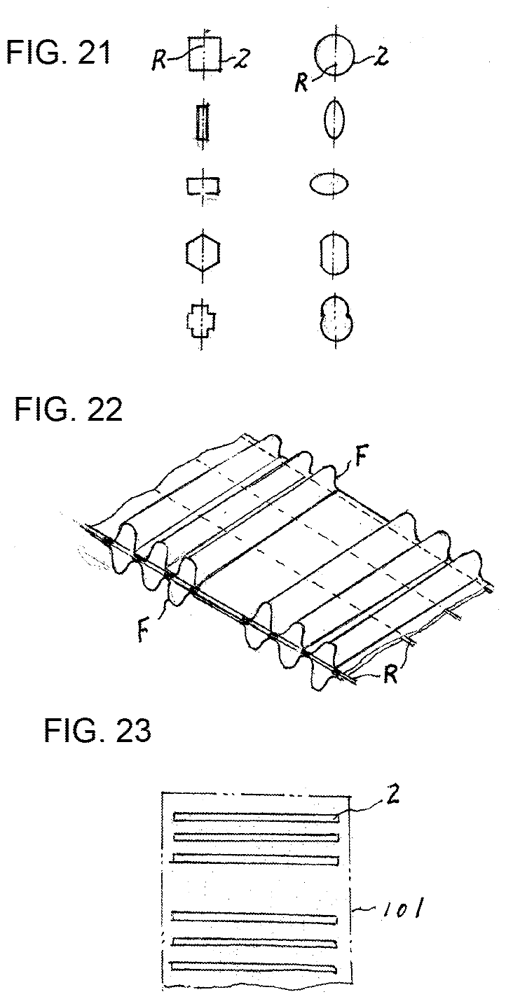

A pattern roll that provides the fixed area formed by this two-dimensional adhesive fixing is shown, and the distribution of the individual protruding surfaces of the pattern roll surface is a grid-like distribution, as shown in FIG. 20. Various individual protruding surface configurations can be used for the fixed area, such as square, circular, elliptical, and variants of these, as shown in FIG. 21.

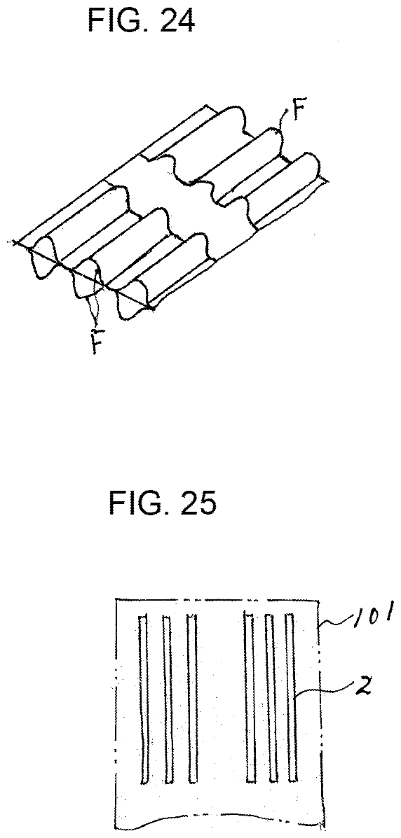

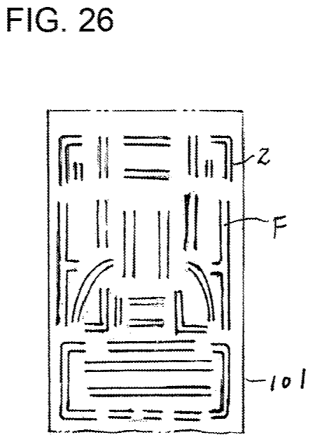

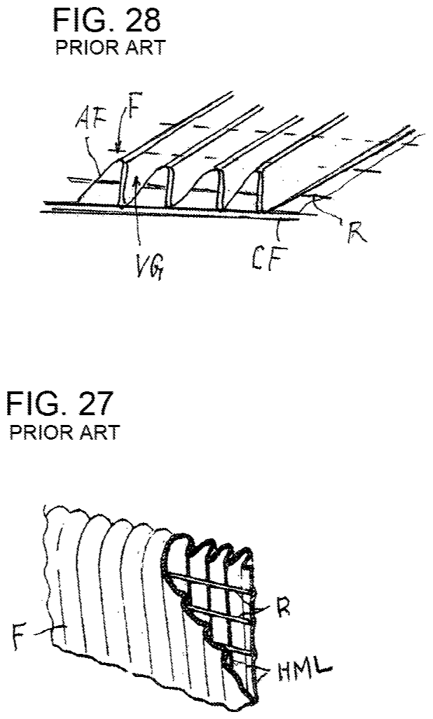

In FIGS. 23 and 25, linear protruding patterns are shown. FIG. 23 shows a horizontal linear protruding pattern, and FIG. 25 is a vertical linear protruding pattern.

The stretchable composite sheet of FIG. 24 is a finished product based on the horizontal linear protruding pattern of FIG. 23, in which the areas between pleats become wide valleys where there are fixed adhesion areas, and there are grooves in the lateral direction.

The stretchable composite sheet in FIG. 26 is a finished product based on the vertical linear protruding pattern in FIG. 25; and when an elastic nonwoven fabric having elastic stretchability in the lateral direction is used as a second sheet, pleats are formed in the longitudinal direction.

A pattern distribution is also possible in which various types of protruding patterns having different configurations are mixed as shown in FIG. 26. In this case, the position and configuration of the pleats and grooves can be set as needed; accordingly, when the stretchable composite sheet is used as a filter element, it is a good idea for the air passage and the washing water passage to have a labyrinthine configuration.

### INDUSTRIAL APPLICABILITY

The present invention provides a stretchable composite member that is pliant, soft, and feels good to the touch, offering flexibility in stretchable composite members used for, for example, body fluid absorbent products such as disposable diapers, medical products such as bandages and protective equipment, and impact- and heat-resistant clothing.

Also, in a stretchable composite member constituting a filter, the shape and height of the breathers (gathers), the shape and width of the grooves, and so forth can be appropriately selected, so that a filter that is suited to the application can be provided. Accordingly, the present invention contributes to the development of the manufacturing industries of related products.

### REFERENCE SYMBOL LIST

AF First sheet-like nonwoven fabric (first coating substrate)
BF Second sheet-like nonwoven fabric (second coating substrate)
R Stretched cord-like elastic member
2 Protruding surfaces of pattern roll
106 Comb-shaped guide

1. A method for manufacturing a stretchable composite member, wherein a coater head whose slot grooves run in an axial direction of a pattern roll is provided to face a pattern surface of the pattern roll, and a hot melt adhesive supplied from a hot melt adhesive supply device is supplied in a thin film state to the pattern surface of the pattern roll, thereby supplying a thin film of hot melt adhesive to only the surface which is protruding;
in a step of transferring the thin film of hot melt adhesive applied to only the protruding surface of the pattern roll from the protruding surfaces of the pattern roll to a surface of a coating substrate,
a first sheet-like nonwoven fabric and a stretched cord-like elastic member are positioned on a protruding surface side of the pattern roll, and the hot melt adhesive is intermittently applied in a travel direction of the stretched cord-like elastic member to the stretched cord-like elastic member and the first sheet-like nonwoven fabric at location where the protruding surface of the pattern roll is present; and
in a subsequent nip roll step,
the stretched cord-like elastic member and the first sheet-like nonwoven fabric, on which intermittent coatings are done in a previous step, and a second sheet-like nonwoven fabric are adhesively fixed, and
the first sheet-like nonwoven fabric and the second sheet-like nonwoven fabric are integrally adhesively fixed along with the hot melt adhesive intermittently applied to the stretched cord-like elastic member at position corresponding to location where the protruding surface of the pattern roll is present;
so that an integrally fixed area of the first sheet-like nonwoven fabric, the stretched cord-like elastic member, and the second sheet-like nonwoven fabric is intermittently distributed so as to correspond to a configuration of the protruding surface.

2. A method for manufacturing a pleated stretchable composite member, wherein a coater head whose slot groove runs in an axial direction of a pattern roll is provided to face a surface of the pattern roll, and a hot melt adhesive supplied from a hot melt adhesive supply device is supplied in a thin film state to the pattern surface of the pattern roll, thereby supplying a thin film of hot melt adhesive to only the surface which is protruding;
in a step of transferring the thin film of hot melt adhesive applied to only the protruding surface of the pattern roll from the protruding surface of the pattern roll to a surface of a coating substrate,
the protruding surface of the pattern roll is in a linear form in a direction perpendicular to a rotation direction of the pattern roll,
a first sheet-like nonwoven fabric and a stretched cord-like elastic member are supplied to a transfer position of the protruding surface of the pattern roll, and
the cord-like elastic member is contact-guided to a lower face of a comb-shaped guide provided between the supplied first sheet-like nonwoven fabric and the supplied cord-like elastic member, so that only the cord-like elastic member is brought into contact with the protruding surface of the pattern roll in front of the transfer position and so that the thin film hot melt adhesive is supplied and applied to only the stretched cord-like elastic member before the hot melt is supplied and applied to the first sheet-like nonwoven fabric; and
in a subsequent nip roll step, the stretched cord-like elastic member and the first sheet-like nonwoven fabric intermittently applied in a previous step and a second sheet-like nonwoven fabric are adhesively fixed,
the first sheet-like nonwoven fabric and the second sheet-like nonwoven fabric are integrally adhesively fixed in a dotted-line pattern along with the hot melt adhesive intermittently applied to the stretched cord-like elastic member at position corresponding to location where the protruding surface of the pattern roll is present;
so that the integrally fixed area of the first sheet-like nonwoven fabric, the stretched cord-like elastic member, and the second sheet-like nonwoven fabric is intermittently distributed in a linear configuration in a direction perpendicular to a travel direction of the stretched cord-like elastic member.

3. A stretchable composite sheet in which a plurality of stretchable elastic members (rubber threads) are adhesively fixed between two nonwoven fabric sheets in which numerous breathers are formed in a lateral direction, thereby forming two of breathers, wherein:
a first sheet-like nonwoven fabric and a second sheet-like nonwoven fabric are integrally adhesively fixed by a hot melt adhesive intermittently applied to a stretched cord-like elastic member at a position corresponding to a location where a protruding surface of a pattern roll is provided;
an integrally fixed area is formed in the first sheet-like nonwoven fabric and the second sheet-like nonwoven fabric is formed;
a plurality of integrally fixed areas are provided in a two-dimensional layout that corresponds to a pattern of protruding surface of the pattern roll; and
the integrally fixed areas are expanded, when viewed in a lateral cross-section, in an extended direction of an entire edge of an application area that corresponds to a protruding surface of the pattern roll.

4. (canceled)
